(19) United States
(12) Patent Application Publication
(10) Pub. No.: US 2009/0253162 A1
(43) Pub. Date: Oct. 8, 2009

(54) METHOD AND APPARATUS FOR ANALYZING SKIN AND HAIR

(75) Inventors: J. Brian Windsor, Austin, TX (US); Robert Eugene Hanes, Austin, TX (US); Damon V. Borich, Austin, TX (US); Jason Neeser, Austin, TX (US); Michael Rasoulian, Austin, TX (US)

Correspondence Address: CHALKER FLORES, LLP, 2711 LBJ FRWY, Suite 1036, DALLAS, TX, 75234, US

(73) Assignee: Reveal Sciences, LLC, Austin, TX (US)

(21) Appl. No.: 12/420,760

(22) Filed: Apr. 8, 2009

Related U.S. Application Data
(60) Provisional application No. 61/043,388, filed on Apr. 8, 2008.

(51) Int. Cl. C12Q 1/02

(52) U.S. Cl. 435/29

(57) ABSTRACT

The present invention includes compositions, methods, and systems for the analysis of skin and hair conditions. The system includes a method and apparatus for analyzing skin and hair samples by taking a sample, identifying desired components of the sample, obtaining an image electronically, storing the image, and analyzing the image utilizing analysis software.

### CROSS-REFERENCE TO RELATED APPLICATIONS

This application claims priority to U.S. Provisional Application Ser. No. 61/043,388, filed Apr. 8, 2008, the entire contents of which are incorporated herein by reference.

### TECHNICAL FIELD OF THE INVENTION

The present invention relates in general to the field of skin and hair analysis, and more particularly, to a novel method and apparatus for identifying biological markers to analyze skin and hair.

### STATEMENT OF FEDERALLY FUNDED RESEARCH

None.

### BACKGROUND OF THE INVENTION

Without limiting the scope of the invention, this invention relates generally to the field of skin and hair analysis, and, more particularly, to the development of a method and apparatus for identifying biological markers for the analysis of skin and hair conditions. Skin and hair analysis is important and greatly desired. Factors such as aging, physical condition, environmental stress, seasonal changes, hormonal fluctuation, biochemical irregularities, and other variables contribute to skin conditions and skin problems. Skin analysis tools assist professionals or individual customers in determining skin type and potential factors contributing to skin conditions. The resulting information allows the customer to choose the most appropriate cosmetic or personal care products in order to maintain or improve skin conditions.

Four general types of methods for skin analysis are currently available. First, questionnaires are used to make a determination of a patient's perceptions of their skin type, condition and needs. The answers to these questionnaires are processed to match a patient's apparent needs with certain pre-determined and pre-packaged skin care products. Second, tape stripping products are used to take a standard visual image of the skin that is compared to a standard visual image of various skin types to determine skin type. Based on the visual matching of the skin type one of products are selected for use. Third, scope or sensors are used to magnify the skin or make determinations of skin water contact, “oiliness” and elasticity.

Again, based on these few physical parameters skin care products are selected. Fourth, 3-D imaging of the skin surface is used to determine skin melanin content, subcutaneous blood flow (by detecting hemoglobin), pore size, skin tone, bacterial content and even skin damage. Again, based on the analysis of these physical parameters, skin care products are selected.

One such system is taught in U.S. Pat. No. 7,211,043, issued to Pruche, et al. for a method, system and device for evaluating skin type. Briefly, the patent is direct to a method, device and system for determining skin type. The method includes a step of applying at least one drop of substance onto a zone of the skin or on a collector member previously in contact with the zone of the skin. The substance can modify at least one physicochemical property of the surface of the zone or of the collector member exposed to the substance. After the drop has disappeared or been removed, the extent of the surface is evaluated and the skin type is determined as a function of this evaluation.

While several methods for analyzing skin are currently available, including imaging and analysis of physical factors, none have used biological markers for determining variables applicable to determining skin and hair health and condition.

### SUMMARY OF THE INVENTION

The needs of the invention set forth above as well as further and other needs and advantages of the present invention are achieved by the embodiments of the invention described herein below. Briefly, the present invention includes compositions, methods and systems for the determination of skin surface biochemical content and characteristics that are not attainable using technology currently available. The system must conform to current technology and methods of use to maximize user compliance. It has been found, remarkably, that a relatively small sample of biochemical skin surface markers serve as surrogates for overall skin condition and treatment options. While hundreds of biochemical parameters could be obtained and explored, the present invention provides both in-depth knowledge, but makes it possible to minimize the parameters that provide maximal results. Furthermore, it was also found that the method and system of the present invention also provide a comprehensive understanding of hair condition.

More particularly, the present invention includes compositions, systems and methods for analyzing skin and hair sample biochemistry comprising: obtaining a skin or hair sample on a capture medium; adding a chemical reagent for identification of specific skin components therein; capturing one or more skin or hair sample images in the visible or non-visible light spectrum; storing the image in an electronic medium; and correlating the presence, absence or amount of a specific skin component with skin condition into a skin condition dataset. In one aspect, the reaction between the sample and the chemical reagent is, e.g., luminescent, calorimetric or fluorescent. The method may also include the step of washing one or more times the sample prior to capturing an image. The capture medium may be integral with a housing that includes the storage medium, or the capture medium may be replaced after every use and the image dates from each use is stored in the storage medium. The skin samples may be obtained by non-invasive methods, including but not limited to tape-stripping, swabs, blot tissue, swipes, wipes, etc. The skin samples obtained may also include invasive methods, including but not limited to punch biopsy, blister fluids, skin-derived lymph, brushing, etc. Hair samples may be obtained by non-invasive methods, including but not limited to cutting of the hair. The chemical reagents are designed or known to react with biomarkers or analytes of the skin or hair.

The images may be captured within the non-visible spectrum light capturing image electronically is accomplished with a reader-like device wherein skin sample placed on cartridge and placed into reader. The non-visible spectrum of light may also be used to capture image electronically is accomplished with a reader-like device wherein skin sample placed into a cuvette. For example, image of the non-visible spectrum light capturing image may be transmitted from the reader to a storage medium by a computer port. The non-visible spectrum light capturing device may capture the image electronically in a portable device. A specific skin condition dataset may be made available on a telecommunications network and the user can access the skin condition dataset from any location.

Another embodiment of the present invention is a system for assessment of skin or hair conditions, comprising: a skin or hair sample substrate for capturing a skin or hair sample; a chemical reagent for identification of one or more skin or hair components that correlates to a skin or hair condition; an image capture device for taking one or more images of the reaction between a chemical reagent and the one or more skin or hair components; capturing the one or more images; storing the one or more images in a memory device; and correlating the levels of component to a skin or a hair condition by determining the presence, absence or amount of a reaction in the sample.

Yet another embodiment of the present invention is an apparatus for analyzing skin and hair samples of a person, comprising: a skin or hair sample capture substrate; a chemical reagent for identification of specific biochemical components therein; an image capture device capable of capturing one or more one image with one or more light sources; and a memory storage device; wherein the image is analyzed for the present, absence or amount of the specific biochemical components from an image captured electronically.

### BRIEF DESCRIPTION OF THE DRAWINGS

For a more complete understanding of the features and advantages of the present invention, reference is now made to the detailed description of the invention along with the accompanying figures and in which:

FIG. 1 shows a test strip of the present invention;

FIG. 2 shows a conceptual rendering of a detection system that shows the insertion of the test strip;

FIGS. 3A and 3B show a test strip before (3A) and after (3B) contact with a skin surface in which the strip has acquired a sample of skin surface components;

FIG. 4 shows a close-up of the test strip with the skin surface components under white light;

FIG. 5A to 5C shows the test strip with the skin surface components under three different lighting conditions that demonstrate the detection of three different skin components and their relative intensity, which translate to the amount of the target measured;

FIG. 6 is a screen shot showing the multi-color analysis and one parameter or indicator measured.

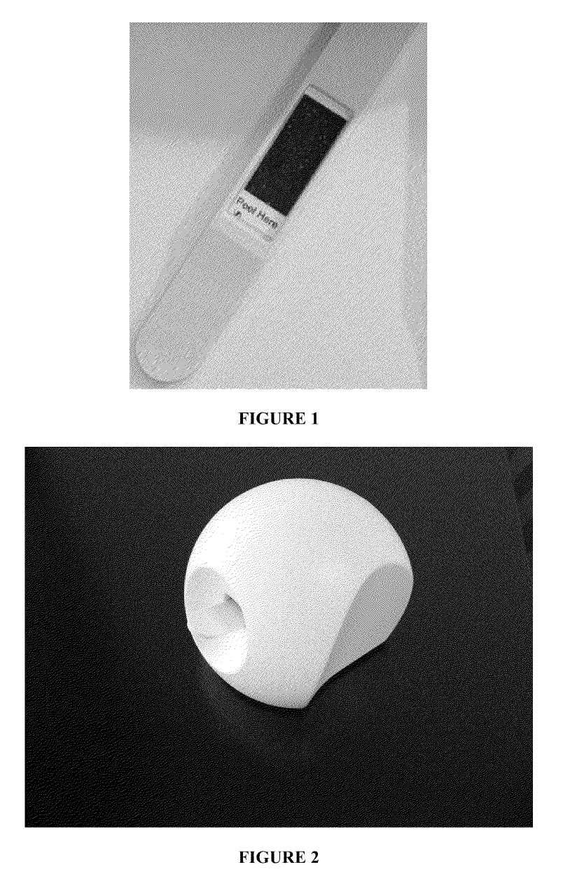

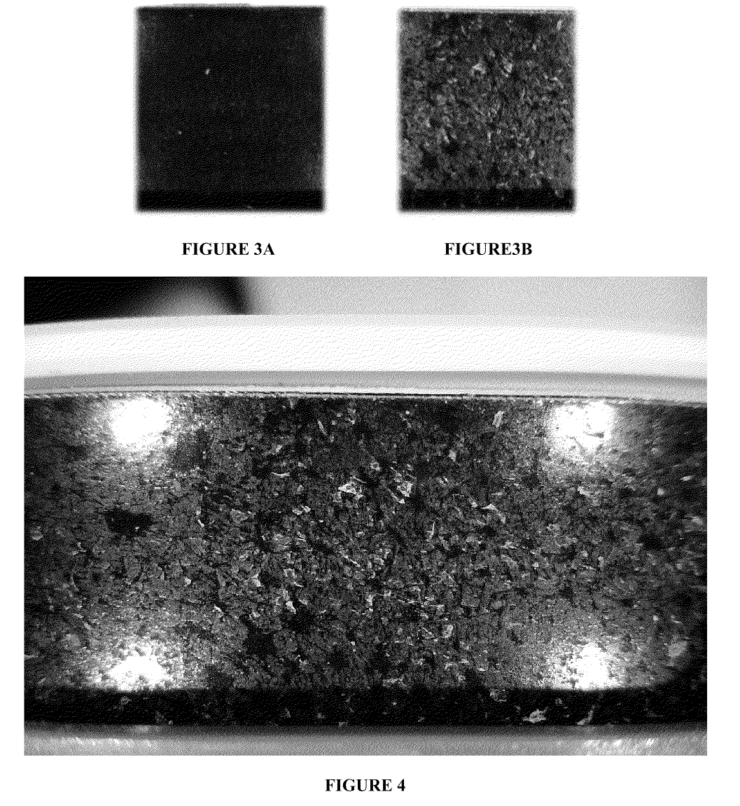

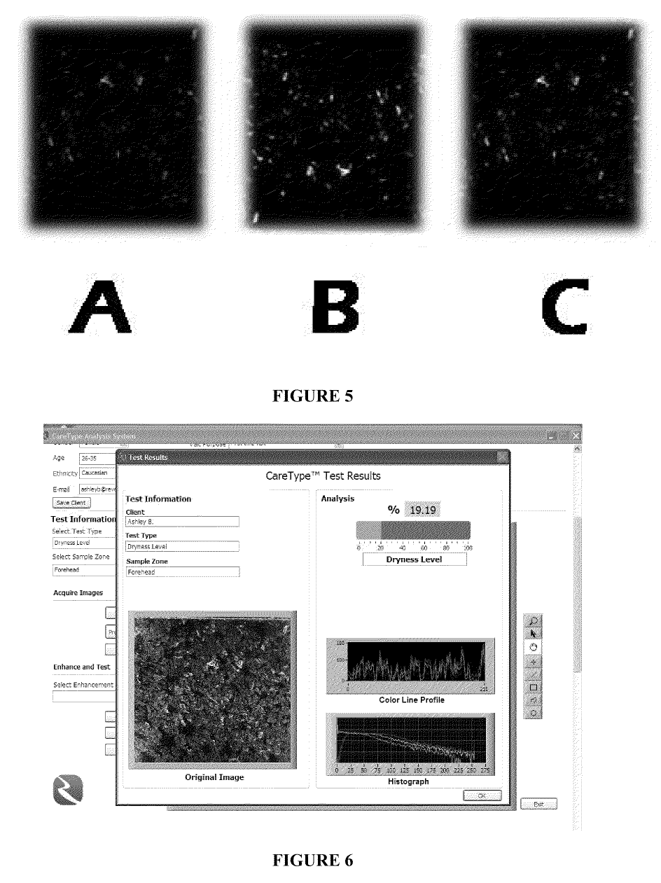

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Generally, all technical terms or phrases appearing herein are used as one skilled in the art would understand to be their ordinary meaning.

There are many potential areas of use for such a skin and hair analysis system: a) medical spa industry, which offers aesthetic services such as laser-therapy, Botox, chemical peels, hair-removal, etc.; b) salons, spas, and resorts that offer products and treatments such as facials, wraps, peels, and full body treatments, etc; and c) health & wellness specialists that tender homeopathy, naturopathy, chiropractic, and herbal medicine; d) dermatologists; e) aestheticians; and f) pharmacy retailers (compounding or retail chains). The present invention provides health care professionals information about the consumer/client/patient's skin or hair that will be useful in choosing the appropriate skin or hair care products to remedy the condition or improve quality of skin and health. Another potential area the skin and hair analysis system would be useful is at the beauty counter of high-end retailers and department stores where personal care and cosmetic products are sold. The invention will also be useful to industry, clinical research companies, and ingredient manufacturers. Most notably, the consumer will benefit.

Current methods for analyzing skin are 1) imaging or 2) determination of physical factors. For example, current methods for determining appearance of skin, fine lines, wrinkles, ageing, sagging and UV damage are mostly visualization of the face by various imaging apparatus. Visual images range from a basic or magnified photograph to three-dimensional (3-D) optically enhanced images. Most of these images can be self-assessed or graded visually by an expert during a consultation in salons, medical spas or by dermatologists. Scopes and sensors are the most widely used imaging devices. Typically they consist of a camera that magnifies skin and pores, and a sensor that measures oil, hydration and elasticity of the skin. Commercial examples are the etude and i-scopeUSB made by the company Moritex. Skin appearance, oiliness, dryness, elasticity, texture, pigmentation, and squames are analyzed.

3-D imaging is more expensive but not much more sophisticated in terms of information provided to the customer/client/patient. Spectral imaging detects melanin and hemoglobin under UV or fluorescent lighting. A software tool and statistical model interpret the information to generate a report that evaluates wrinkles, spots, pore size, skin tone (evenness), bacterial content and UV spots indicative of sun damage or oxidation. Examples of commercially available systems are the VISIA by Canfield Imaging Systems or the ClarityPro by Moritex. There are several problems associated with these imaging systems, such as lighting and positioning. A shift in position or slight variations in lighting may cause differences that appear to be skin conditions, but are not. The present invention overcomes these problems because the skin sample is taken from the person and inserted into the USB or reader device so that lighting is consistent and correctly positioning the face is irrelevant.

Various other methods exist to determine physical factors such as skin hydration, elasticity, and barrier function. Skin hygrometers are type of apparatus that measure electrical capacitance and conductance of skin in order to determine the skin's relative hydration. Hydration has also been measured via spectroscopy, including acoustic, infrared, and Nuclear Magnetic Resonance (NMR), and corneometry. Skin elasticity is often measured using a ballistometer, dermal torque meter, or by pinch recoil. Skin barrier function and water evaporation are usually measured by transepidermal water loss (TEWL). Most of these methods for evaluating skin parameters are used in clinical efficacy trials and would not work at beauty counter in a retail distributor or in the medical spa or salon. The present invention provides for such application. The user can determine efficacy of particular ingredients and correlate results to definite biomarkers.

Current state-of-art is that skin type is generally described by purely physical factors such as dry, oily, normal or combination skin. Combination skin encompasses both oily and dry skin patches. Current methods for determining skin type range from customers completing inclusive questionnaires to non-invasive methods. Questionnaires are typically used at the counter in department stores or for online product sales. For example, companies such as Chanel, Clinique and Olay use questionnaires to assess customer skin type and recommend skin care products and cosmetics based on those skin types. Consumers answer questions about skin sensitivity to sunlight; skin color (fair, light, medium, dark, yellow, pink, etc.); eye and hair color; pore size; breakout tendency or if acne-prone; oily versus dry; visible wrinkles; and so forth. The answers to these questions define skin characteristics and help the consumer chose appropriate products.

Currently available means for determining skin condition do not provide adequate information to determine the cause of skin conditions. This approach is very subjective and relies on the customer's answers. Preconceived notions and inaccurate information will vary the answers and are not very helpful in assessing actual conditions. Most of the skin conditions or biomarker are non-visible and can't be readily determined by visual assessment or answering a few subjective questions. The present invention reveals the underlying biomarkers and the causes of skin conditions or problems, which provides for specific personal care. For instance, red skin can be caused by many different conditions. Dry skin, itchy skin and flakiness are all symptoms of several different problems or diseases that could be caused by ceramide breakdown, loss of natural moisturizing factors, irritants, soap, allergies, bacteria, oxidation, sun damage and/or eczema, psoriasis, etc. Currently, consumers are forced use products by trial and error until something works.

Tape-stripping is a non-invasive approach that permits a direct quantitative and qualitative assessment of biomarkers from the skin surface and stratum corneum (SC). Examples of tape-stripping products commercially available are D-Squame (CuDerm), Sebutape (CuDerm), various adhesive and mailing tapes (3M), and cyanoacrylate resin. Samples are taken by applying the adhesive tape to a target area of skin in a manner sufficient to isolate an epidermal sample adhering to the adhesive tape. Layers of the SC can be sequentially removed by repeated application of pieces of adhesive tape. The epidermal sample contains biomarkers that correlate to skin conditions. Currently, tape-stripping is used in research or for marketing.

These methods give varying degrees of qualitative information, but do not offer any way of detecting specific biomarkers or skin analytes linked to various skin conditions. For instance, an apparatus can determine that skin is dry or less elastic. However, the apparatus does not give the consumer a reason for the dryness or loss of elasticity. It is necessary to determine various biomarkers that correlate to skin conditions in order to make such a determination. The present invention correlates biological markers to skin or hair conditions.

The current invention detects biomarkers that correlate to specific skin conditions and provides quantitative data for skin assessments unlike 3-D imaging machines, sensors, and scopes. Consumers, salon professionals, medical spa professionals, and personal care product manufacturers are in need of consumer/product feedback to ensure best product usage, compliance and effectiveness. The current invention provides information to the consumer which enables more product specialization and better product selection. Less wasteful spending on product trial-and-error is inevitable as customers are specifically determined or diagnosed according to biological molecules rather than superficial surveys or general physical qualities. This will ultimately improve consumer confidence in a product, brand support for manufacturers, and improve skin health.

Abundance of biomarkers correlating to skin conditions exemplify why it is currently difficult to coordinate products to consumer skin conditions. Stratum corneum is the outer layer of the skin that interacts with the environment. Biomarkers naturally occurring in the stratum corneum are natural moisturizing factors (NMFs), proteins, enzymes, lipids, fatty acids, ceramides, and cytokines. The presence of aldehydes, carbonyl proteins, vitamins, surfactants, metals, pollutants, porphyrins, and bacteria are indicative of various skin conditions. Imbalance of naturally occurring biomarkers or the presence of one or more analytes correlate to skin conditions including, but not limited to dryness, itchiness, flaking, scaling, roughness, wrinkles, elasticity, age spots, bumps, redness, and inflammation. These skin conditions are implicated in several skin problems or diseases, such as oxidative or sun-damage, dehydration, acne, irritation, aging, wrinkles, inflammation, rosacea, eczema, psoriasis, and allergic or contact dermatitis.

Natural moisturizing factors molecules are generated by hydrolysis of the protein filaggrin into free amino acids (serine, glycine, arginine, ornithine, citrulline, alanine, histidine), urocanic acid, pyrrolidone carboxylic acid, lactate, sugars, urea, chloride, sodium, potassium, ammonia, uric acid, glucosamine, creatine, calcium, magnesium, phosphate, citrate and formate. NMFs are implicated in skin conditions such as dryness, flaking, scaling, inflammation, and ageing.

Numerous proteins are found in stratum corneum, such as keratin, comeodesmosin, loricrin, suprabasin, desmoglein, and others. It is further well known in the art that oxidative stress causes: 1) oxidative cleavage of proteins; 2) direct oxidation of amino acids; 3) carbonyl groups introduced into proteins via reactions with aldehydes derived from degradation of lipid peroxides. Increased carbonyl protein levels correlate to dryness, scaling, roughness, wrinkles, loss of elasticity, and ageing. Furthermore, aldehydes in cigarette smoke cause damaging carbonyl formation in skin.

Vitamins, derivatives, forms and complexes. UV exposure and oxidation cause a decrease in the human SC's natural anti-oxidants such as vitamins A, C, and E (in various derivative, forms and complexes). The major form of vitamin A is an alcohol (retinol), but can also exist as an aldehyde (retinal), as an acid (retinoic acid), as an ester (retinyl palmitate) and as beta-carotene. Vitamin A is known to improve condition of skin; but retinol causes inflammation of the skin. Vitamin C (in its various derivatives, forms and complexes) is an anti-oxidant that also enhances the synthesis of collagen. Vitamin B3 (Niacin/Niacinamide) helps the skin retain moisture and upregulates ceramide synthesis. Vitamin D deficiency may occur with use of sunscreen because sunlight is necessary to convert Vitamin D into a bioavailable form. Vitamin D3 is produced photochemically in the skin from 7-dehydrocholesterol. Vitamin K is known to repair dark, under eye circles and bruises as well as healing spider veins.

Enzymes found in the stratum corneum include, but are not limited to beta-glucocerebrosidase, phospholipases, acid phosphatase, serine proteases: trypsin (chymotrypsin), cholesterol sulfatase, sphingomyelin deacylase, prosaposin, transglutaminase, peptide methionine sulfoxide reductases, and acid ceramidase. Phospholipases: Type IV cPLA(2)-a (calcium dependent) and type I or II sPLA(2) (secretory) are found in the skin. Type II sPLA(2) is implicated in inflammation. Increased acid phosphatase activity correlates to dry, itchy skin. Reduced trypsin activity correlates to dry, itchy skin and scaling. Altered levels of prosaposin, a regulator of sphingolipid metabolism, are implicated in dry, itchy skin as well as roughness, bumps and inflammation. Peptide methionine-5-sulfoxide reductase is a unique repair enzyme indicative of skin-oxidation and cell-ageing.

Cholesterol esters and cholesterol sulfate are part of the stratum corneum barrier function. Cholesterol sulfate accumulates when deficient in steroid sulfatase enzyme (recessive X-linked ichthyosis—genetic disease); induces transcription of transglutaminase; inhibits serine proteases involved in desquamation. Transglutaminase activity correlates to dryness and scaling of the skin.

Many analytes or biomarkers interact with each other in various synthesis and degradation pathways. For example, Ceramide EOS (Cer(OS)) main ceramide component of stratum corneum. It contains an omega-hydroxy fatty acid ester-linked to linoleic acid and amide-linked to sphingosine. Free linoleic acid is necessary to maintain skin barrier function, and as such altered levels correlate to dry skin, scaling and inflammation. Furthermore, decreased levels of free sphingosine reflect decreased levels of ceramide and diminished acid ceramidase activity which cause scaling of the skin. Another example, a decrease in CER(EOS) levels causes an increase in sphingomyelin deacylase, which competes with sphingomyelinase for the ceramide precursor sphingomyelin, causing an increase in sphingosyl phosphoryl choline. Sphingosyl phosphoryl choline stimulates proliferation and up-regulation of plasminogen activator. Elevated levels of sphingomyelin deacylase and sphingosyl phosphoryl choline correlate to dry, itchy skin as well as roughness, bumps, and inflammation.

The presence of unusual species are indicators of skin conditions. Presence of co-hydroxy acid, stimulates ceramide production in the epidermis, and can be correlated to scaling and inflammation. Ceramide(AS) is an unusual species and is correlated to dry, itchy, scaling, roughness, bumps and inflammation. Triglycerides, short-chain saturated fatty acids and unsaturated fatty acids are sebaceous contaminants whose presence may serve to disrupt barrier organization at skin surface correlated to dry skin. Phospholipids should not be present in healthy stratum corneum.

Cytokines are known to cause wrinkles, redness, and inflammation. Several interleukins have been detected on the skin surface. For example: IL-8, IL-6, IFN-γ, IL-4, IL-13 cause inflammation; TNF-α correlates to scaling, roughness, redness, inflammation (Benson, et al., 2006); and IL-1α and IL-IRA (receptor antagonist) involved in epidermal signaling and indicative of ageing and inflammation.

Glucocorticoids delay barrier recovery and lead to dry, itchy skin.

Surfactants are known to bind to stratum corneum proteins and cause dry, itchy skin, scaling, roughness, loss of elasticity, bumps, and inflammation. They are usually used in soaps, syndets, and detergents. Sodium lauryl sulfate (SLS)/sodium dodecyl sulfate (SDS) and sodium lauroyl ether sulfate (SLES) are anionic surfactants and bind proteins of the SC. Sodium lauroyl isethionate (SLI) is also anionic but binds 1/5 as strongly to SC proteins as SLS/SDS. Lauroyl amido propyl betaine is amphoteric and binds SC proteins to a much lesser degree than anionic surfactants. Other surfactants known to bind SC proteins are monoalkyl phosphate, sodium cocoyl isethionate, cocamidopropyl betaine (CAPB), and alkyl polyglucoside (APG).

Analytes: Metals such as nickel are irritants that can cause bumps, redness and irritation. (Nyren, Kuzmina, & Emtestam, 2003)

Acne is caused by various factors, including excessive sebum and poor desqammation of the stratum corneum. Indicators are bacterial contamination (P. acnes) and porphryins secreted by bacteria (coproporphyrin I, coproporphyrin III, and protoporphyrin). These are metal-free fluorescent porphyrins that can be easily detected on the skin surface.

Hormones: Dihydrotestosterone (DHT) is a hormone that has also been correlated to oily skin, bumps, redness, inflammation. A decrease in the hormone estrogen causes dryness and wrinkles. This condition often occurs during the aging process.

Biomarkers, Epidermis, Dermis, etc. Samples from a tissue can be isolated by any number of means well known in the art. Invasive methods for isolating a sample include the use of needles, for example during blood sampling, as well as biopsies of various tissues.

There are biomarkers in epidermis, dermis or other tissue samples that correlate to skin conditions and diseases. For example, matrix metalloproteinases (MMPs) and their inhibitors (MMPIs) are highly regulated molecules found in the dermal layer. MMPs are known to play a role in pathological conditions such as inflammation and wound healing. There are several families of MMPs, one group are called collagenases (MMP-1, MMP-8, and MMP-13), which can cleave interstitial collagens I, II, and III at a specific site as well as degrade other ECM and non-ECM molecules. Specifically, MMP-1 is known to degrade collagen I, collagen II, collagen III, gelatin, and proteoglycans. MMP-8 is known to degrade collagens I, II, III, V, VII, IX, and gelatin. MMP-13 in known to degrade collagens I, II, III, IV, IX, X, XIV, fibronectin, and gelatin. The presence of certain MMPs and MMPIs, as well as variations in basal level can be biomarkers correlated to aging and an increase in wrinkles and roughness as well as a loss of elasticity.

Similarly, the presence and relative levels of glycosaminoglycans (GAG) and proteoglycans found in the dermal layer can be correlated to the roughness and elasticity of the skin. Hyaluronan is found in varying biological forms both in the epidermal and dermal layers of the skin and can be correlated to wrinkling of the skin. Biglycan, decorin, and fibronectin play a significant role in roughness, wrinkles, and ageing of the skin.

Molecules that form the core structure of the dermal layer, such as elastin and collagen are major players in ageing of the skin, leading to roughness, wrinkles, and loss of elasticity.

The present invention is used to obtain quantitative data as well as qualitative imaging; broad spectrum of test measures, such as novel biochemical assays, measure custom markers by product, and design by skin condition; product performance indications; product selection information for consumer/product matching with a quick; simple system; and at a lower cost than 3-D imaging.

By identifying non-visible skin health markers, more information is given to the professional and consumer enabling more product sales, more product specialization, improving consumer confidence in a product, improving skin health and increasing brand support for manufacturers.

In one embodiment, the invention provides a method enabling analysis of skin and hair samples of a person, the method including a step of taking a skin or hair sample. A chemical reagent for identification of specific components in the sample may be added. At least one image is taken with one or more light sources; and non-visible spectrum light captures the image electronically. A memory device will store the image, which can be analyzed and displayed immediately or stored for later processing and display. In one embodiment, the present invention comprises a reader device, disposable test trips or cartridges, and a computer-implemented system to provide a product feedback method.

Skin samples are taken by tape-stripping method and incorporated into a carrier such as a cartridge or test strip. Cartridges or test strips will detect various analytes or biomarkers that correlate with various skin conditions, including but not limited to:

1) aldehydes, carbonyl proteins, and decreases in vitamin E levels are indicative of skin oxidation and products containing anti-oxidants, sun protection, vitamins should be recommended;

2) a depletion in NMF, ceramides, and varying levels of skin surface enzymes are indicative of dry skin and products such as moisturizers, soaps, and ceramide production enhancers should be recommended;

3) the presence of porphyrins or excessive sebum (oil) coupled with poor desquamation are indicative of acne and products containing salicylic acid, benzoyl peroxide, Retin-A along with specific skin care regimens should be recommended;

4) surfactants, metals (Ni²⁺), pollutants and allergens can cause redness or irritation of the skin and the appropriate chemical and natural peels, masks, and detoxification products should be recommended;

5) ceramides, carbonyls, aldehydes, and collagen levels all relate to skin aging and wrinkles and products with collagen-enhancing treatments, peptides, sunscreen, anti-oxidants, Botox, or surgery should be recommended; and

6) new analytes, specifically requested analytes, or ingredients will be incorporated into new cartridges (can correlate analyte to new products being developed for research and product feedback).

At least one or more of these cartridges are inserted into a reader device. Images are captured of the skin sample. Another embodiment is to incorporate chemicals into the cartridge or test strip that will react with the skin sample. The software will have algorithms to correlate biomarkers in the skin sample with skin conditions. A report will be generated for the professional or consumer who can then recommend various products relating to the skin condition. Propionibacterium acnes produce propionic acid and it is one of the primary aggravating conditions that results in acne in humans. Propionic acid may be detected with a synthetic receptor, or probing a change of pH with colorimetric dyes. In another embodiment, a synthetic receptor may be combined with a dye to detect propionic acid in an indicator displacement assay.

In one embodiment, a detection scheme as described above is combined with the sampling substrate. In certain embodiments the detection scheme includes a functionalized adhesive composition able to react to propionic acid or an associated change in pH. The sample substrate is then loaded into the cartridge and imaged, providing for acne analysis.

In other embodiments, structured illumination tomography could be used to obtain depth information of the sample and construct a three-dimensional, tomographic representation of the sample. The technique includes illuminating the sample at different frequencies of light and obtaining multiple images of the sample under the different illuminations. These images are then combined and processed with mathematical algorithms to extract dimensional information and ultimately create a tomographic representation. This method can be used to accurately assess, for example, wrinkle depth and width. This technique generally uses projecting the light source as a sinusoidal wave pattern of varying frequencies and phase shifts to vary penetration depth into the sample. Different wavelengths of light may also be used to obtain information necessary for the tomographic processing.

Different light sources can be used to illuminate and/or interact with the sample, including a DLP (digital light processing) system that usesa digital micromirror device. A tunable liquid crystal filter can also be used to selectively use certain wavelengths of light.

In one embodiment, all components of the above specification are used together. Different angles of light are used to illuminate the surface and different types of light are used at those angles in sequence including U.V., white, infrared and other spectra. The surface is optically interrogated for surface structure information and pattern recognition is used in conjunction with the surface structure information and chemical changes including reactions between dyes and analytes, capture media and sample media, types of light and photosensitive powders and liquids. The specifications and setting of each of these components manipulate how the sample is optically interrogated. A multitude of images are then collected and analyzed. In another embodiment structured illumination tomography is used in conjuction with the above methodology. In other embodiments the above methodology is used in conjunction with principle component analysis to analyze the images.

In one embodiment a tape strip is used to sample one area of skin treated with a sunscreen product that has had adequate time and been thoroughly rubbed in. A second tape strip is used against an untreated area of the skin. Each tape strip is then placed in the cartridge and dosed with U.V. light. The amount of background fluorescence is then imaged and the two images are compared to measure the amount of U.V. blockage on the skin. In one embodiment a test strip with an adhesive coating and a white fluorescent background is used. The greater the sunscreen in the skin from the removed layer of the stratum corneum the less the background fluorescence will be captured in the image. In other embodiments any cosmetic product is tested in the manner above to demonstrate the degree of U.V. protection.

In one example, a sample is dosed with phosphorescent powder in preparation for an image capture. The U.V. light charges the sample with photons in order to activate phosphorescence. Powdered phosphorescent materials typically rest on surfaces, whereas liquid phosphorescent materials will settle into crevices. Powders and liquids are utilized to collect 3 dimensional and structural information about the sample. For example, when taking the image of a fingerprint after using solid phosphorescent materials, the ridgelines are visualized. Conversely, when a liquid phosphorescent material was applied to a finger, the liquid settled into the crevices permitting a ‘negative’ image of the fingerprint. These dual modes for visualizing a fingerprint illustrates the data provided for image recognition and interrogating the surface, e.g., dual mode used to detect an analyte. In the same or separate embodiments the U.V. light can be used to sterilization the sample prior to processing.

Infrared light (I.R) is used to probe the nature of the organic functional groups present on the skin. Infrared spectroscopy typically is used to uniquely characterize the nature of compounds on the skin. In certain embodiments, image interrogation with IR radiation, coupled with image processing provides a way to detect unique chemical compounds on the skin.

In one example the sample media reacts with the capture medium. Sample media may be comprised of innate or exogenous compounds such as biomarkers found on the skin or components of an adhesive composition that react with a capture media comprising for example synthetic receptors in the example of biomarker detection, or other chemical compositions reactive with preloaded adhesive components.

In another example, the sample media and or the capture media changes with light. This can be accomplished with NLO (non-linear optical) materials that change their birefringence when interrogated with different wavelengths of light. The resulting change in optical signal distinguishes the sample depending upon the originating wavelength. This method provides additional data for identifying skin conditions.

In certain embodiments, skin markers may be tagged with dyes that change optical properties upon the formation of the complex. Such dyes may be calorimetric, or fluorescent. In some embodiments a complex is formed, as a result of one or more of the following forces single, or working in concert: Van der Waals forces, hydrogen bonding, hydrophobic effects, ionic bonding, charge transfer complexes or reversible covalent bonds driven by equilibrium. In another embodiment, the formation of the complex results in the displacement of a third molecule or ion. Such a displacement may be identified directly, or through the interaction of a third dye.

Dye Properties. The paired dye system may include calorimetric or fluorescent dyes. Signaling methods could include visible ‘turn on’ or ‘turn off’ visible to the human eye, fluorescence resulting from the stimulation of UV light, chemiluminescence or phosphorescence.

The Primary Dye. In one embodiment the primary dye may not change its absorption, emission or otherwise signaling the presence of the primary analyte. Under such conditions, the primary dye may be a synthetic receptor with whose properties are designed to chelate the primary analyte and the secondary analyte, but not necessarily act as a dye in the conventional sense. In another embodiment the primary dye modulates its optical properties upon the binding of the primary or secondary analyte. For instance, preloading the primary dye with the secondary or signaling analyte could change the color of the primary dye. Upon contact with the primary analyte, the color of the primary dye could revert to the color before binding of the signaling analyte.

For the purposes of clarity, the primary dye functions to bind the primary and secondary analyte. Upon binding of the primary analyte, the secondary analyte is released either stoichiometrically, or in a controlled rate or quantity proportional to the amount of primary analyte detected. In this way, the detection system can be biased to respond to a given analyte threshold level for a qualitative type test. Further the system can be calibrated to reveal a quantitative result that could be coupled with a detection device.

The Secondary Dye. The role of the secondary dye is to amplify the signal of the primary analyte through the dye's interaction with the secondary analyte. Such a change may be brought about through the use of metal chelating dyes such as cresolphthalein complexone whose color appears in the presence of calcium, for example. The response of the dye to calcium is much greater to the unaided eye than would be for an organic analyte. This system allows for greater response by using a more sensitive secondary analyte for triggering the detection system.

Binding Properties of the Dyes. The intermolecular forces responsible for binding analyte include, but are not limited to and may be in combination of: hydrogen bonding, ion pairing, hydrophobic effects, charge transfer complexes, reversible covalent bonds, pi stacking.

Anchoring Agents. Anchoring agents provide additional flexibility for the platform through the immobilization of the detection reagents. The anchoring agents may comprises handles through which to covalently attach the dyes, or may possess sufficient binding forces to effectively immobilize the dye and prevent leaching when exposed to fluid flow. Examples include synthetic and natural polymers and derivatives thereof comprising functional groups such as amines, alcohols, aldehydes, ketones, esters that may be used as covalent handles or non covalent binding. Further, the choice of polymer backbone can be made by the skilled artisan based on the present disclosure based on the material's ability to interact with various analytes, or dyes. Polyallylamine, polyacrylics, guar, HP guar, CHMHP guar, alginic acid, xanthan, and chitosan, glucosamine.

Typical strategies for covalent attachment can be used, e.g., using dehydrating agent to make amide bonds from carboxylic acids and amines. The coupling strategies are not limited to amides, which are provided as a non-limiting example.

### Example 1

### Method for Detection of Calcium on the Skin

A swab comprising an integrated buffer was used to sample the skin. The buffer was used to rinse the swab, then the mixture was transferred to the test platform which comprises a dipstick with an analysis membrane and a testing zone for imaging. The membrane comprises a competing paired dye system with dyes with different binding affinities toward calcium. Polyallylamine was conjugated to EDTA and an indicator which stripped calcium metal ions from the effluent stream. The swab buffer comprises a dye that responds to calcium, that once stripped of the metal results in a color change. The stripped dye is captured by a secondary line for imaging. The ratio of the indicator in the EDTA line to the sequestered dye permits a algorithm to analyze the mixture for the presence of different metals ions based upon the ratio of the two dyes. Examples of dyes that function in such a system include arseanzo III, cresolphthalein complexone and calmagite. One skilled in the art would recognize other indicators and chelating agents that could be used to image and identify the presence of calcium based on the present disclosure.

### Example 2

### Method for Detection of Alpha-Hydroxy Acids

A swab comprising an integrated buffer was used to sample the skin. The buffer was used to rinse the swab, then the mixture was transferred to the test platform which comprises a dipstick with an analysis membrane and a testing zone for imaging. The membrane comprises a competing paired dye system with dyes with different binding affinities toward alpha hydroxyacids. Polyallylamine was conjugated to 2-formylphenyl boronic acid which was labeled with alizarin complexone and the mixture was applied to nitrocellulose as a stripe. In the presence of an alpha hydroxyacid, the dye is displaced. Then the dye travels down the nitrocellulose membrane where it is sequestered by a secondary dye or synthetic receptor coupled to another polyallylamine stripe. The secondary dye or synthetic receptor amplifies the alizarin complexone by turning on fluorescences.

### Example 3

### Method for Detecting MMP Enzymes on the Skin Surface

A swab comprising an integrated buffer was used to sample the skin. The buffer was used to rinse the swab, then the mixture was transferred to the test platform which comprises a dipstick with an analysis membrane and a testing zone for imaging. The membrane comprises a competing paired dye system with dyes with different peptide sequences with varying affinities toward different MMP's. Depending upon the MMP present (e.g., MMP1-14), the peptide labeled with a dye is cleaved, then released to interact with the secondary dye. The secondary dye or synthetic receptor amplifies the severed dye through fluorescence quenching, enhancement or calorimetric change from the sequestering agent.

In one embodiment the swab in the previous examples is integrated on the tape strip platform. In accordance with another exemplary embodiment, the present invention will comprise of a USB connected to a computer screen, disposable test strips or cartridges, and software. In accordance with another exemplary embodiment, the present invention will comprise a portable reader device, disposable test strips or cartridges, and software. In accordance with another exemplary embodiment, the present invention will comprise a portable reader device, disposable test strips or cartridges, and software. In accordance with another exemplary embodiment, skin sample will be taken by various other invasive or non-invasive methods. In accordance with another exemplary embodiment, hair samples will be taken by various other invasive or non-invasive methods. In accordance with another exemplary embodiment, chemical reagents comprising color-changing, calorimetric, fluorescence or chemiluminescence technology will be incorporated.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Ananthapadmanabhan, K. P., Moore, D. J., Subramanyan, K., Misra, M., & Meyer, F. (2004). Cleansing without compromise: the impact of cleansers on the skin barrier and the technology of mild cleansing. Dermatologic Therapy, 17, 16-25.
Ashkenazi, H., Malik, Z., Harth, Y., & Nitzan, Y. (2003). Eradication of Propionibacterium acnes by its endogenic porphyrins after illumination with high intensity blue light. FEMS Immunology and Medical Microbiology, 35, 17-24.
Benson, N. R., Papenfuss, J., Wong, R., Motaal, A., Tran, V., Panko, J., et al. (2006). An Analysis of Select Pathogenic Messages in Lesional and Non-Lesional Psoriatic Skin Using Non-Invasive Tape Harvesting. The Journal of Investigative Dermatology, 126, 2234-2241.
Berlett, B. S., & Stadtman, E. R. (1997). Protein Oxidation in Aging, Disease, and Oxidative Stress. The Journal of Biological Chemistry, 272, 20313-20316.
Borelli, C., Merk, K., Schaller, M., Jacob, K., Vogeser, M., Weindl, G., et al. (2006). In Vivo Porphyrin Production by P. acnes in Untreated Acne Patients and its Modulation by Acne Treatment. Acta Dermato-Venereologica, 86, 316-319.
Doherty, T. M., Asotra, K., Pei, D., Uzui, H., Wilkin, D. J., Shah, P. K., et al. (2002). Therapeutic developments in matrix metalloproteinase inhibition. Expert Opinion on Therapeutic Patents, 12 (5), 665-707.
Fujita, H., Hirao, T., & Takahashi, M. (2007). A simple and non-invasive visualization for assessment of carbonylated protein in the stratum corneum. Skin Research and Technology, 13, 84-90.
Fulmer, A. W., & Kramer, G. J. (1986). Stratum corneum lipid abnormalities in surfactant-induced dry scaly skin. The Journal of Investigative Dermatology, 86, 598-602.
Harding, C. R. (2004). The stratum corneum: structure and function in health and disease. Dermatologic Therapy, 17, 6-15.
Knuutinen, A., Kokkonen, N., Risteli, J., Vahakangas, K., Kallioinen, M., Salo, T., et al. (2002). Smoking affects collagen synthesis and extracellular matrix turnover in human skin. British Journal of Dermatology, 146, 588-594.
Kobayashi, Y., Iwai, I., Akutsu, N., & Hirao, T. (2008). Increased carbonyl protein levels in the stratum corneum of the face during winter. International Journal of Cosmetic Science, 30, 35-40.
Mazereeuw-Hautier, J., Redoules, D., Tarroux, R., Chaveron, M., Salles, J. P., Simon, M. F., et al. (2000). Identification of pancreatic type I secreted phospholipase A2 in human epidermis and its determination by tape stripping. British Journal of Dermatology, 142, 424-431.
Morhenn, V. B., Chang, E.-Y., & Rheins, L. A. (1999). A noninvasive method for quantifying and distinguishing inflammatory skin reactions. Journal of the American Academy of Dermatology, 41, 687-692.
Nakagawa, N., Sakai, S., Matsumoto, M., Yamada, K., Nagano, M., Yuki, T., et al. (2004). Relationship between NMF (Lactate and Potassium) content and the physical properties of the stratum corneum in healthy subjects. Journal of Investigative Dermatology, 122, 755-763.
Nyren, M., Kuzmina, N., & Emtestam, L. (2003). Electrical impedance as a potential tool to distinguish between allergic and irritant contact dermatitis. The Journal for the American Academy of Dermatology, 48, 394-400.
Ogawa, F., Sander, C. S., Hansel, A., Oehrl, W., Kasperczyk, H., Elsner, P., et al. (2006). The Repair Enzyme Peptide Methionine-S-Sulfoxide Reductase is Expressed in Human Epidermis and Upregulated by UVA Radiation. The Journal of Investigative Dermatology, 126, 1128-1134.
Primavera, G., & Berardesca, E. (2005). Sensitive skin: mechanisms and diagnosis. International Journal of Cosmetic Science, 27, 1-10.
Rawlings, A. V., & Harding, C. R. (2004). Moisturization and skin barrier function. Dermatologic Therapy (17), 43-48.
Rawlings, A. V., Canestrari, D. A., & Dobkowski, B. (2004). Moisture technology versus clinical performance. Dermatologic Therapy, 17, 49-56.
Roguet, R., Cohen, C., Robles, C., Courtellemont, P., Tolle, M., Guillot, J. P., et al. (1998). An interlaboratory study of the reproducibility and relevance of episkin, a reconstructed human epidermis, in the assessment of cosmetics irritancy. Toxicology In Vitro, 12, 295-304.
Thiele, J. J., & Ekanayake-Mudiyanselage, S. (2007). Vitamin E in human skin: Organ-specific physiology and considerations for its use in dermatology. Molecular Aspects of Medicine, 28, 646-667.
Thiele, J. J., Traber, M. G., & Packer, L. (1998). Depletion of human stratum corneum vitamin E: an early and sensitive in vivo marker of UV induced photo-oxidation. The Journal of Investigative Dermatology, 110, 756-761.
Visse, R., & Nagase, H. (2003). Matrix metalloproteinases and tissue inhibitors of metalloproteinases. Circulation Research, 92, 827-839.

What is claimed is:

1. A method of analyzing skin and hair sample biochemistry comprising:
obtaining a skin or hair sample on a capture medium;
capturing one or more skin or hair sample images in the visible or non-visible light spectrum;
storing the image in an electronic medium; and
correlating the presence, absence or amount of a specific skin component with skin condition into a skin condition dataset.

2. The method of claim 1, wherein the reaction between the sample is luminescent, fluorescent or calorimetric.

3. The method of claim 1, further comprising the step of washing one or more times the sample prior to capturing an image.

4. The method of claim 1, wherein the capture medium is integral with a housing that includes the storage medium.

5. The method of claim 4, wherein the capture medium is replaced after every use and the image dates from each use is stored in the storage medium.

6. The method of claim 1, wherein the skin samples are obtained by non-invasive methods, including but not limited to tape-stripping, swabs, blot tissue, swipes, wipes, etc.

7. The method of claim 1, wherein the skin samples are obtained by invasive methods, including but not limited to punch biopsy, blister fluids, skin-derived lymph, brushing, etc.

8. The method of claim 1, wherein the hair samples are obtained by non-invasive methods, including but not limited to cutting of the hair, etc.

9. The method of claim 1, further comprising a dry chemical reagent designed or known to react with biomarkers or analytes of the skin or hair.

10. The method of claim 1, wherein the image is obtained with a reader-like device, wherein the skin sample is on a capture medium that is integral with a housing comprising a storage medium and the housing is placed into reader.

11. The method of claim 1, wherein the image is transmitted electronically to and from the storage medium in a housing.

12. The method of claim 1, wherein the image is captured by a portable device.

13. The method of claim 1, wherein the specific skin condition dataset is available on a telecommunications network and the user can access the skin condition dataset from any location.

14. A system for assessment of skin or hair conditions, comprising:
a skin or hair sample substrate for capturing a skin or hair sample;
a chemical reagent for identification of one or more skin or hair components that correlates to a skin or hair condition, wherein the chemical reagent is provided in dry form;
an image capture device for taking one or more images of the reaction between a chemical reagent and the one or more skin or hair components;
capturing the one or more images;
storing the one or more images in a memory device; and
correlating the levels of component to a skin or a hair condition by determining the presence, absence or amount of a reaction in the sample.

15. The system of claim 14, wherein the reaction between the sample is luminescent, fluorescent or calorimetric.

16. The system of claim 14, wherein the sample is washed one or more times prior to capturing an image.

17. The system of claim 14, wherein the capture medium is integral with a housing that includes the storage medium.

18. An apparatus for analyzing skin and hair samples of a person, comprising:
a skin or hair sample capture substrate;
a chemical reagent for identification of specific biochemical components therein;
an image capture device capable of capturing one or more one image with one or more light sources; and
a memory storage device; wherein the image is analyzed for the present, absence or amount of the specific biochemical components from an image captured electronically.

19. A system for testing residual versus innate products and by products of the skin comprising:
a skin or hair sample capture substrate;
a chemical reagent for identification of specific biochemical components therein;
an image capture device capable of capturing one or more one image with one or more light sources; and
a memory storage device; wherein the image is analyzed for the present, absence or amount of the specific biochemical components from an image captured electronically.

20. A system for measuring the amount of U.V. blockage of a cosmetic comprising:
a skin sample capture substrate with a fluorescent background;
an image capture device capable of capturing one or more one image with one or more ultraviolet light sources;
a memory storage device; wherein the image is analyzed for background fluorescence from an image captured electronically.

21. A method of analyzing skin and hair sample biochemistry comprising:
obtaining a skin or hair sample on a capture medium;
capturing one or more skin or hair sample images in the visible or non-visible light spectrum using light emitting diodes;
storing the image in an electronic medium; and
correlating the presence, absence or amount of a specific skin component with skin condition into a skin condition dataset.
